(19) United States
(12) Patent Application Publication
(10) Pub. No.: US 2014/0364460 A1
(43) Pub. Date: Dec. 11, 2014

(54) USE OF FATOSTATIN FOR TREATING CANCER HAVING A p53 MUTATION

(71) Applicant: THE TRUSTEES OF COLUMBIA UNIVERSITY IN THE CITY OF NEW YORK, New York, NY (US)

(72) Inventors: William Allen Freed-Pastor, Canfield, OH (US); Carol Prives, New York, NY (US); Timothy Osborne, Merritt Island, FL (US)

(73) Assignee: THE TRUSTEES OF COLUMBIA UNIVERSITY IN THE CITY OF NEW YORK, NEW YORK, NY (US)

(21) Appl. No.: 14/373,147

(22) PCT Filed: Jan. 18, 2013
(86) PCT No.: PCT/US2013/022326

§ 371 Date: Jul. 18, 2014

Related U.S. Application Data
(60) Provisional application No. 61/588,158, filed on Jan. 18, 2012.

(51) Int. Cl. A61K 31/4439; C12Q 1/68; A61K 45/06

(52) U.S. Cl. CPC A61K 31/4439; A61K 45/06; C12Q 1/68
USPC 514/342; 546/270.4; 435/6.14; 435/6.11; 435/6.12

(57) ABSTRACT

Fatostatin, a recently described inhibitor of SREBP activation, significantly reduces the level of mutant p53 binding to the HMG-CoA reductase gene promoter. Further, fatostatin treatment had a dramatic effect on normalizing the abnormal 3D morphology of 3 strains of breast cancer cells: MDA-468 cells, MDA-231 cells, and SKBR3 cells. The results show a functional interaction with SREBPs as being critical for mutant p53-mediated upregulation of the mevalonate pathway genes. At a clinical level, inhibition of the mevalonate pathway, either alone or in combination with other therapies, offers a novel, safe and much needed therapeutic option for tumors bearing mutant p53.

### CROSS-REFERENCE TO RELATED APPLICATIONS

This application claims the benefit of Provisional Application No. 61/588,158 filed Jan. 18, 2012, which is incorporated in its entirety.

### STATEMENT OF GOVERNMENTAL INTEREST

This invention was made with Government support under Contract No. NCI CA87497 awarded by the United States Department of Health and Human Services National Institutes of Health. The Government has certain rights in the invention.

### BACKGROUND

Mutations in p53 are a frequent event in cancer. Despite the huge diversity in the genes implicated in tumorigenesis, the p53 transcription factor—encoded by the human gene TP53—stands out as a key tumor suppressor and a master regulator of various signaling pathways involved in this process. The many roles of p53 as a tumor suppressor include the ability to induce cell cycle arrest, DNA repair, senescence, and apoptosis, to name only a few. Indeed, TP53 mutations were reported to occur in almost every type of cancer at rates varying between 10% (e.g., in hematopoietic malignancies), 25-40% (of breast cancers, but some studies report that two-thirds of all breast cancers display p53 mutations), and close to 100% (e.g., in high-grade serous carcinoma of the ovary.) The evolution of a normal cell toward a cancerous one is a complex process, accompanied by multiple steps of genetic and epigenetic alterations that confer selective advantages upon the altered cells. Inactivation of the p53 tumor suppressor is a frequent event in tumorigenesis. In most cases, the p53 gene is mutated; giving rise to a stable mutant protein whose accumulation is regarded as a hallmark of cancer cells. Mutant p53 proteins not only lose their tumor suppressive activities but often gain additional oncogenic functions that endow cells with growth and survival advantages.

Aberrant forms of human p53 are associated with poor prognosis, more aggressive tumors, metastasis, and short survival rates in multiple tumor types. Despite massive research efforts and the very impressive progress made over the past several decades, full molecular understanding of cancer still remains a major challenge to the biomedical community. At a clinical level, inhibition of the mevalonate pathway, either alone or in combination with other therapies, offers a novel, safe and much needed therapeutic option for tumors bearing mutant p53. Therefore, there is a need for developing treatments for cancers having p53 mutations.

### SUMMARY OF THE INVENTION

Certain embodiments are directed to methods for treating or preventing cancer, or reducing or eliminating precancerous cells or a benign tumor that have a mutated p53 gene or that express a mutant p53 protein or mRNA encoding a mutant p53 protein in a subject, by administering to the subject a therapeutically or prophylactically effective amount of a sterol regulatory element binding protein (SREBP) cleavage activating protein inhibitor, such as fatostatin or an analogue thereof, alone, or in combination with a statin. Identifying a subject that will respond to treatment is the result of obtaining a biological sample of the cancer, the precancerous cells or the cells of a benign tumor from the subject and determining if these sample cells have the mutant p53 gene or express the mutant p53 protein or an mRNA encoding the mutant p53 protein. If the mutant p53 gene or expression of the mutant p53 protein or mRNA encoding mutant p53 is detected, then the subject will respond to treatment with the SREBP cleavage activating protein inhibitor and is treated. Biological samples in certain embodiments include, but are not limited to, tumor biopsies, urine, blood, cerebrospinal fluid, sputum, serum, stool, or bone marrow. In certain embodiments, therapeutically effective amounts of the SREBP cleavage activating protein inhibitor fatostatin or an analogue thereof range from about 0.1 mg/kg to about 150 mg/kg per administration with as many administrations per day as are needed to achieve the desired result, and for as long as is needed.

In the above method, the cancer to be treated includes cancer cells selected from the group consisting of lung cancer, digestive and gastrointestinal cancer, gastrointestinal stromal tumors, gastrointestinal carcinoid tumors, colon cancer, rectal cancer, anal cancer, bile duct cancer, small intestine cancer, stomach (gastric) cancer, esophageal cancer, gall bladder cancer, liver cancer, pancreatic cancer, appendix cancer, breast cancer, ovarian cancer, renal cancer, cancer of the central nervous system, skin cancer, lymphomas, choriocarcinomas, head and neck cancers, osteogenic sarcomas, and blood cancer.

In methods of treatment, the inhibitor can be administered by any means that is shown to achieve the desired result, including orally, by injection (i.p., subcutaneous, etc.), parenterally, by inhalation, topically, rectally, nasally, buccally, vaginally or via an implanted reservoir. Fatostatin or an analogue thereof can also be administered locally to the site of the cancer or benign tumor.

Some embodiments are directed to pharmaceutical formulations comprising a SREBP cleavage activating protein inhibitor such as fatostatin or an analogue thereof, alone, or in combination with one or more statins as well as kits comprising them. In certain embodiments, a pharmaceutical formulation may comprise fatostatin or an analogue thereof, in an amount 0.1 mg/kg to about 150 mg/kg alone, or in combination with a statin. In some embodiments, the amount of statin is below 80 mg, between 80 mg and 150 mg, between 150 mg and 250 mg, between 250 and 350 mg, and between 350 mg and 1 gram. The amount of therapeutic agent depends on many factors including bioavailability, route of administration, the aggressiveness of the cancer, and whether the cancer is a tumor or circulating cancerous cells. The statin may be selected from the group consisting of lovastatin, simvastatin, pravastatin, fluvastatin, atorvastatin, and cerivastatin.

Certain embodiments of the present invention are directed to methods for determining if a subject with cancer or precancerous lesions or a benign tumor, will respond to treatment (i.e. if the patient and the cancer will respond to treatment) with a SREBP cleavage activating protein inhibitor such as fatostatin or an analogue thereof by (i) obtaining a sample of the cancer cells, precancerous cells or benign tumor cells from the subject, (ii) assaying the cells in the sample for the presence of a mutated p53 gene or a mutant form of p53 protein or a biologically active fragment thereof or an mRNA encoding a mutant form of p53, and (iii) if the cells have the mutated p53 gene or mutant form of the p53 protein or an mRNA encoding a mutant form of p53, then determining that the subject will respond to treatment with the inhibitor or combinations. Yet other embodiments are directed to a method of preventing recurrence of cancer, precancerous lesions or a benign tumor or methods of preventing cancer in a subject at high risk of developing cancer comprising a p53 protein or gene mutation, by administering fatostatin, alone or together as a combination treatment with a statin.

These and other features, aspects, and advantages of the present invention will become better understood with regard to the following description, appended claims, and accompanying figures.

### BRIEF DESCRIPTION OF THE DRAWINGS

The following figures form part of the present specification and are included to further demonstrate certain embodiments of the present invention. The invention may be better understood by reference to one or more of these drawings in combination with the detailed description of specific embodiments presented herein.

FIG. 1. MDA-468.shp53 cells were treated with fatostatin (20 μM) and subjected to ChIP analysis. Data are presented as mean+−SD of six independent experiments. **p<0.01

FIG. 2. MDA-231.shp53 cells were grown in 3D cultures for 8 days and treated with (A) DMSO, (B) Fatostatin 2 μM or (C) Fatostatin 20 μM. Drugs were added on day 1. Representative DIC images are shown. Scale bar, 200 μm.

FIG. 3. Fatostatin inhibits SKBR3 cell growth in 3D Culture. SKBR3 cells were grown in 3D cultures for 8 days treated with (A) DMSO, (B) Fatostatin 2 μM or (C) Fatostatin 20 μM. Drugs were added on Day 1. Representative Differential Interference Contrast (DIC) images are shown. Scale bar, 200 μm.

FIG. 4. Fatostatin inhibits MDA-468 cell growth in 3D Culture. MDA-468.shp53 cells were grown in 3D cultures for 10 days treated with (A) DMSO, (B) Fatostatin 2 μM or (C) Fatostatin 20 μM. Drugs were added on Day 1. Representative Differential Interference Contrast (DIC) images are shown. Scale bar, 200 μm.

### DETAILED DESCRIPTION

### 1. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N. Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N. Y. (1990); Principles of Neural Science, 4th ed., Eric R. Kandel, James H. Schwart, Thomas M. Jessell editors. McGraw-Hill/Appleton & Lange: New York, N. Y. (2000). Unless defined otherwise, all technical and scientific terms used herein have the same meaning as commonly understood by one of ordinary skill in the art.

As used herein, “administering” a SREBP cleavage activating protein inhibitor e.g., fatostatin alone or in combination with a statin, may be performed using any of the various methods of delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, introccularly, via local delivery, subcutaneously, intraadisposally, intraarticularly, intrathecally, into a cerebral ventricle, intraventricularly, intratumorally, into cerebral parenchyma or intraparenchymally or microinjection.

As used herein, the terms “animal,” “patient,” or “subject” include mammals, e.g., humans, dogs, cows, horses, kangaroos, pigs, sheep, goats, cats, mice, rabbits, rats, and transgenic non-human animals. The preferred animal, patient, or subject is a human.

As used herein, the term “Sterol Regulatory Element-Binding Proteins (SREBPs” means transcription factors that bind to the sterol regulatory element DNA sequence TCACNCCAC. Mammalian SREBPs are encoded by the genes SREBF1 and SREBF2. SREBPs belong to the basic-helix-loop-helix leucine zipper class of transcription factors. Unactivated SREBPs are attached to the nuclear envelope and endoplasmic reticulum membranes. PARA NO.

As used herein, the term “fatostatin” means a molecule that specifically binds to a binding partner of SREBP localized in the endoplasmic reticulum of cells called SREBP cleavage activating protein, or “SCAP”. The binding of fatostatin to SCAP prevents the posttranslational processing and maturation of SREBP in the endoplasmic reticulum, a critical step required for nuclear translocation of SREBP, the master regulator of gene expression in the mevalonate pathway. Blocking SCAP inhibits SREBP transcription factors and therefore inhibits the mevalonate and fatty acid biosynthesis pathways. SCAP is a protein that in humans is encoded by the SCAP gene. The chemical name for fatostatin is 125B11, 2-Propyl-4-(4-(p-tolyl)thiazol-2-yl) pyridine, It is also referred to as an SREBP Processing Inhibitor II, and is commercially available from EMD4Biosciences as product 341329 Fatostatin. The following references describe fatostatin synthesis, metabolism and certain uses. Krepinsky et al., Articles in Pres S. Am J Physiol Renal Physiol (Oct. 26, 2011). doi:10.1152/ajprenal.00136.2011; SREBP-1 Activation by Glucose Mediates TGFβ Upregulation in Mesangial Cells; A Small Molecule That Blocks Fat Synthesis By Inhibiting the Activation of SREBP, Shinji Kamisuki et al. in Chemistry & Biology 16, 882-892, Aug. 28, 2009; Synthesis and Evaluation of Diarylthiazole Derivatives That Inhibit Activation of Sterol Regulatory Element-Binding Proteins, Shinji Kamisuki, J. Med. Chem. 2011, 54, 4923-4927.

The term, “kit” as used herein, means any manufacture (e.g., a package or container) comprising at least one reagent, e.g., a SREBP cleavage activating protein inhibitor such as fatostatin and/or in combination with a statin. In certain embodiments, the manufacture may be promoted, distributed, or sold as a unit for performing the methods of the present invention.

A “subject” or “patient” is a mammal, typically a human, but optionally a mammalian animal of veterinary importance, including but not limited to horses, cattle, sheep, dogs, and cats.

A “therapeutic agent” is an inhibitor of an SREBP transcription factor or an inhibitor of an SCAP protein, which regulates SREBP processing, and therefore inhibits the mevalonate pathway, such as fatostatin or fatostatin analogues as herein described.

A “therapeutically effective amount” of a therapeutic agent is an amount that achieves the intended therapeutic effect of reducing or eliminating the cancerous cells, precancerous cells or benign tumor cells that express a mutant form of p53 protein or a p53 gene mutation or an mRNA encoding a mutant form of p53 protein in a subject thereby treating them. The full therapeutic effect does not necessarily occur by administration of one dose and may occur only after administration of a series of doses. Thus, a therapeutically effective amount may be administered in one or more administrations.

A “prophylactically effective amount” of a drug is an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations.

An “effective amount” of an agent is an amount that produces the desired effect.

The term “p53” as used herein refers to both p53 protein and the TP53 gene; “p53 mutations” refers to mutations in the p53 protein and p53 gene.

The term “TP53” as used herein refers to the gene encoding p53 protein.

The term “p53 protein” as used herein refers to a tumor suppressor protein that in humans is encoded by the TP53 gene. p53 is crucial in multicellular organisms, where it regulates multiple cellular processes such as cell cycle arrest, cell death, senescence, metabolic pathways and other outcomes thereby acting as a tumor suppressor that is involved in preventing cancer. p53 is also known as UniProt name: Cellular tumor antigen p53, Antigen NY-CO-13, Phosphoprotein p53, Transformation-related protein 53 (TRP53), Tumor suppressor p53.

The term “mutant form of p53 protein” is used herein as any protein other than wild-type p53 protein.

The terms “polypeptide” and “protein” are used interchangeably as a generic term referring to native protein, fragments, peptides, or analogs of a polypeptide sequence. Hence, native protein, fragments, and analogs are species of the polypeptide genus.

The terms “treat” or “treatment” refer to both therapeutic treatment and prophylactic or preventative measures, wherein the object is to prevent or slow down (lessen) an undesired physiological change or disorder, such as the development, progression or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having cancer and those with benign tumors or precancerous lesions that have a mutant p53 gene. “Treating” cancer in a patient refers to taking steps to obtain beneficial or desired results, including clinical results. For purposes of this invention, beneficial or desired clinical results include, but are not limited to alleviation or amelioration of one or more symptoms of the cancer; diminishing the extent of disease; delaying or slowing disease progression; amelioration and palliation or stabilization of the disease state.

The term “cancer” is intended to include any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, lung cancer (e.g., non-small cell lung cancer); digestive and gastrointestinal cancers such as colorectal cancer, gastrointestinal stromal tumors, gastrointestinal carcinoid tumors, colon cancer, rectal cancer, anal cancer, bile duct cancer, small intestine cancer, and stomach (gastric) cancer; esophageal cancer; gallbladder cancer; liver cancer; pancreatic cancer; appendix cancer; breast cancer; ovarian cancer; renal cancer (e.g., renal cell carcinoma); cancer of the central nervous system; skin cancer; lymphomas; choriocarcinomas; head and neck cancers; osteogenic sarcomas; and blood cancers. As used herein, a “tumor” comprises one or more cancer cells or benign cells or precancerous cells.

A precancerous condition (or premalignant condition) is a generalized state associated with a significantly increased risk of cancer. If left untreated, these conditions may lead to cancer. A premalignant lesion is a morphologically altered tissue in which cancer is more likely to occur than its apparently normal counterpart.

The term “sample” as used herein includes any biological specimen obtained from a subject. Samples include, without limitation, whole blood, plasma, serum, red blood cells, white blood cells (e.g., peripheral blood mononuclear cells), saliva, urine, stool (i.e., feces), tears, nipple aspirate, lymph, fine needle aspirate, any other bodily fluid, a tissue sample (e.g., tumor tissue) such as a biopsy of a tumor, and cellular extracts thereof. In some embodiments, the sample is whole blood or a fractional component thereof such as plasma, serum, or a cell pellet. In certain embodiments, the sample is obtained by isolating circulating cells of a solid tumor from a whole blood cell pellet using any technique known in the art. As used herein, the term “circulating cancer cells” comprises cells that have either metastasized or micro metastasized from a solid tumor and includes circulating tumor cells, and cancer stem cells. In other embodiments, the sample is a formalin fixed paraffin embedded (FFPE) tumor tissue sample, e.g., from a solid tumor.

A nucleic acid sample can be obtained from a subject using routine methods. Such samples comprise any biological matter from which nucleic acid can be prepared. As non-limiting examples, suitable samples include whole blood, serum, plasma, saliva, cheek swab, urine, or other bodily fluid or tissue that contains nucleic acid. In one embodiment, the methods of the present invention are performed using whole blood or fractions thereof such as serum or plasma, which can be obtained readily by non-invasive means and used to prepare genomic DNA. In another embodiment, genotyping involves the amplification of a subject's nucleic acid using PCR. Use of PCR for the amplification of nucleic acids is well known in the art (see, e.g., Mullis et al., The Polymerase Chain Reaction, Birkhauser, Boston, (1994). Generally, protocols for the use of PCR in identifying mutations and polymorphisms in a gene of interest are described in Theophilus et al., “PCR Mutation Detection Protocols,” Humana Press (2002). Further protocols are provided in Innis et al., “PCR Applications: Protocols for Functional Genomics,” 1st Edition, Academic Press (1999). Applicable PCR amplification techniques are described in Ausubel et al., Current Protocols in Molecular Biology, John Wiley & Sons, Inc., New York (1999); Theophilus et al., “PCR Mutation Detection Protocols,” Humana Press (2002); and Innis et al., “PCR Applications: Protocols for Functional Genomics,” 1st Edition, Academic Press (1999). General nucleic acid hybridization methods are described in Anderson, “Nucleic Acid Hybridization,” BIOS Scientific Publishers (1999). Amplification or hybridization of a plurality of transcribed nucleic acid sequences (e.g., mRNA or cDNA) can also be performed using mRNA or cDNA sequences arranged in a microarray. Microarray methods are generally described in Hardiman, “Microarrays Methods and Applications: Nuts & Bolts,” DNA Press (2003) and Baldi et al., “DNA Microarrays and Gene Expression: From Experiments to Data Analysis and Modeling,” Cambridge University Press (2002).

### 2. Overview

It has now been discovered that fatostatin, a recently described inhibitor of SREBP activation (Kamisuki, Shinji et al., 2009), significantly reduced the level of mutant p53 binding to the HMG-CoA reductase gene promoter. Further, fatostatin treatment had a dramatic effect on normalizing the abnormal 3D morphology of 3 strains of breast cancer cells: MDA-468 cells, MDA-231 cells, and SKBR3 cells. The results prove a functional interaction with SREBPs as being critical for mutant p53-mediated upregulation of the mevalonate pathway genes.

The evolution of a normal cell toward a cancerous one is a complex process, accompanied by multiple steps of genetic and epigenetic alterations that confer selective advantages upon the altered cells. Inactivation of the p53 tumor suppressor is a frequent event in tumorigenesis. In most cases, the p53 gene is mutated; giving rise to a stable mutant protein whose accumulation is regarded as a hallmark of cancer cells. Cancers having p53 mutations have been reported to occur in almost every type of cancer at rates varying between 10%-100%. Therefore, it is important to determine the role of mutant p53 and its potential as a target for cancer therapy.

### 3. Background

The TP53 gene, which encodes the p53 protein, is the most frequent target for mutation in tumors, with over half of all human cancers exhibiting mutation at this locus (Vogelstein et al., 2000). Wild-type p53 functions primarily as a transcription factor and possesses an N-terminal transactivation domain, a centrally located sequence specific DNA binding domain, followed by a tetramerization domain and a C-terminal regulatory domain (Laptenko and Prives, 2006). In response to a number of stressors, including DNA damage, hypoxia and oncogenic activation, p53 becomes activated to promote cell cycle arrest, apoptosis or senescence thereby suppressing tumor growth. It also plays many additional roles including regulating cellular metabolism (Muller et al., 2009).

Unlike most tumor suppressor genes, which are predominantly inactivated as a result of deletion or truncation, the majority of mutations in TP53 are missense mutations, a few of which cluster at “hotspot” residues in the DNA binding core domain (Petitjean et al., 2007), while the N- and C-terminal domains of this protein are relatively spared from mutation (Hussain and Harris, 1998; Soussi and Lozano, 2005; Unger et al., 1993). In contrast to wild-type p53, which under unstressed conditions is a very short-lived protein, these missense mutations lead to the production of full-length p53 protein with a prolonged half-life (Davidoff et al., 1991; Rotter, 1983). While many tumor-derived mutant forms of p53 can exert a dominant-negative effect on the remaining wild-type allele, serving to abrogate the ability of wild-type p53 to inhibit cellular transformation, the end result in many forms of human cancer is frequently loss of heterozygosity (LOH), where the wild-type version of p53 is lost and the mutant form is retained, suggesting that there is a selective advantage conferred by losing the remaining wild-type p53, even after one allele has been mutated (Brosh and Rotter, 2009).

There is substantial evidence that certain mutants of p53 can exert oncogenic, or gain-of-function, activity independent of their effects on wild-type p53. In vivo models, in which mice harboring two tumor-derived mutants of p53 (equivalent to R175H and R273H in humans) that were substituted for the endogenous wild-type p53 locus within the mouse genome, display an altered tumor spectrum as well as more metastatic tumors (Lang et al., 2004; Olive et al., 2004). The mutational status of p53 has been shown to predict poor outcomes in multiple types of human tumors, including breast cancer, and certain mutants of p53 associate with an even worse prognosis (Olivier et al., 2006; Petitjean et al., 2007). Mutant p53 has also been demonstrated to lead to increased survival, invasion, migration and metastasis in preclinical breast cancer models (Adorno et al., 2009; Muller et al., 2009; Stambolsky et al., 2010). Despite these findings, mutant p53-induced phenotypic alterations in mammary tissue architecture have not been fully explored.

The association between mutated p53 protein and TP53 and cancer has been widely studied for most tumor sites in most human ethnic groups (Varley, Hum Mutat 2003; 21:313-20; Royds et al. Cell Death Differ 2006; 13:1017-26, Savage et al. Pediatr Blood Cancer 2007; 49:28-33, Ueda et al. Gynecol Oncol 2006; 100:173-8; Ignaszak-Szczepaniak et al. Oncol Rep 2006; 16:65-7; Wang-Gohrke et al. Br J Cancer 1999; 81:179-83; Wu et al. Cancer Res 2006; 66:8287-92). Different single polymorphisms and haplotypes are associated with different risk increments. The risk for Li-Fraumeni syndrome (multisite cancer syndrome) that involves a germline mutation in p53 increases risk of cancer 100-fold for men and 1000-fold for women. Thus, there is a great need for methods of treating cancer having mutated p53 protein and TP53.

p53 is a frequent target for mutation in mammalian tumors and previous studies have revealed that missense mutant p53 proteins can actively contribute to tumorigenesis. p53 mutations are usually thought to occur is 25-40% of breast cancers, but some studies report that two-thirds of all breast cancers display p53 mutations (Lai et al. (2004) Breast Cancer Res. Treat., 83: 57-66). Aberrant forms of human p53 are associated with poor prognosis, more aggressive tumors, metastasis, and short survival rates in multiple tumor types (Mitsudomi et al., Clin Cancer Res 2000 October; 6(10):4055-63; Koshland, Science (1993) 262:1953), (Petijean et al. 2007).

PCT/US 11/55488 application, incorporated herein by reference, includes the results of experiments showing that:

(i) Depletion of endogenous mutant p53 from breast cancer cells is sufficient to induce a phenotypic reversion in 3D culture from a cancerous morphology to a more normal hollow-lumen acinar morphology. Functional transactivation domains are necessary for mutant p53 to disrupt acinar morphogenesis;

(ii) Mutant p53 upregulates seventeen genes that encode enzymes in the mevalonate pathway;

(iii) The effects of mutant p53 on breast cancer morphology are mediated through the mevalonate pathway. HMG-CoA reductase inhibitors mimic the phenotypic effects of mutant p53 depletion in 3D culture thereby causing the cancer cells to revert to normal morphology or result in a more profound phenotypic effect (i.e. cell death). The normalizing phenotypic effects following downregulation of mutant p53 can be recapitulated by inhibiting critical enzymes in the mevalonate pathway. This normalization can be reversed by supplementing breast cancer cells depleted of mutant p53 with two key intermediate metabolites produced by this pathway, specifically mevalonic acid (MVA) and mevalonic acid 5-phosphate (MVAP). Thus, flux through the mevalonate pathway is both necessary and sufficient for the phenotypic effects of mutant p53 on breast cancer morphogenesis in 3D culture. HMG-CoA reductase inhibitors mimic the phenotypic effects of mutant p53 depletion in breast cancer cells;

(iv) In vivo mouse data shows that treatment with simvastatin reduced tumor size after 21 days of treatment by about 40%;

(v) Not only HMG-CoA reductase, but several downstream enzymatic steps in the mevalonate pathway are involved in the ability of mutant p53 to prevent normal morphological behavior of breast cancer cells in 3D culture conditions; and

(vi) Patient data shows that TP53 mutation correlates with high levels of sterol biosynthesis genes in human tumors.

### 4. Summary of Experimental Results and Embodiments of the Invention

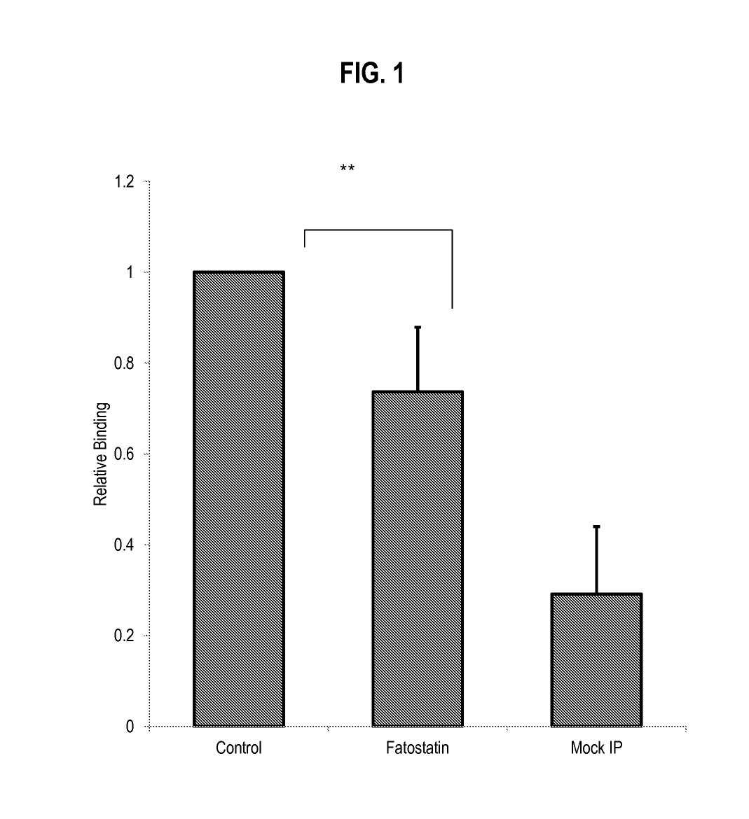

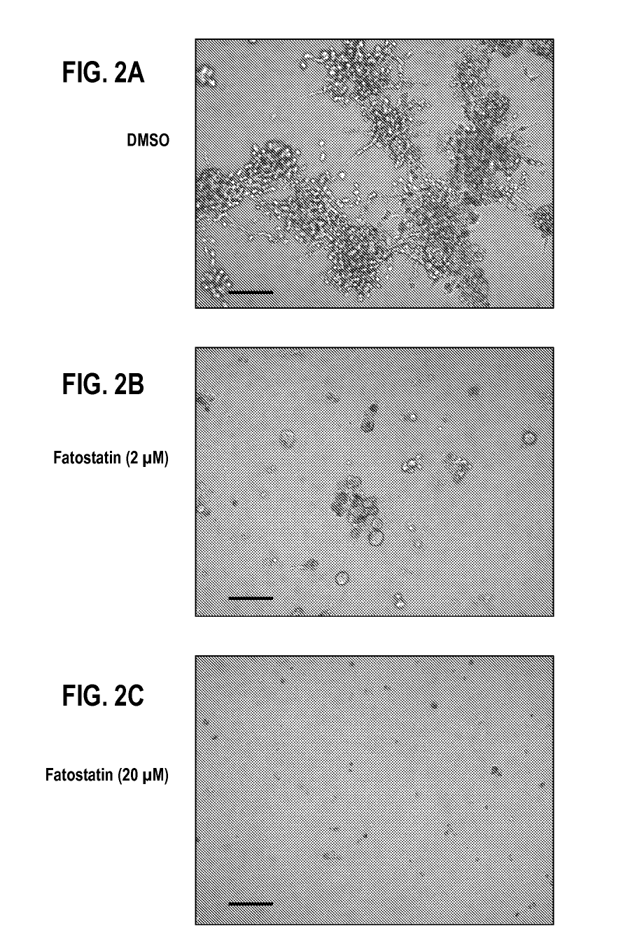

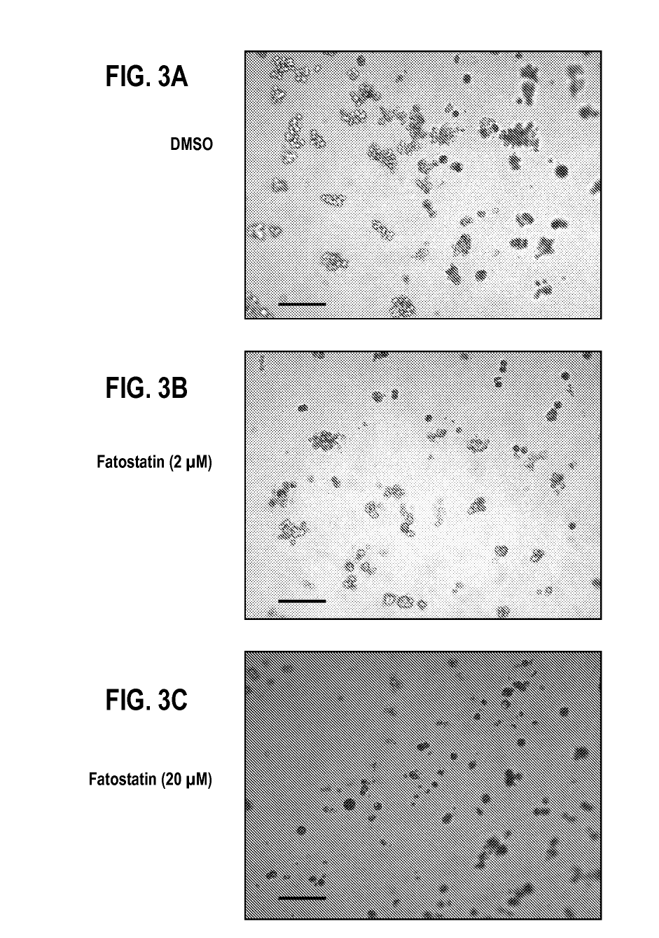

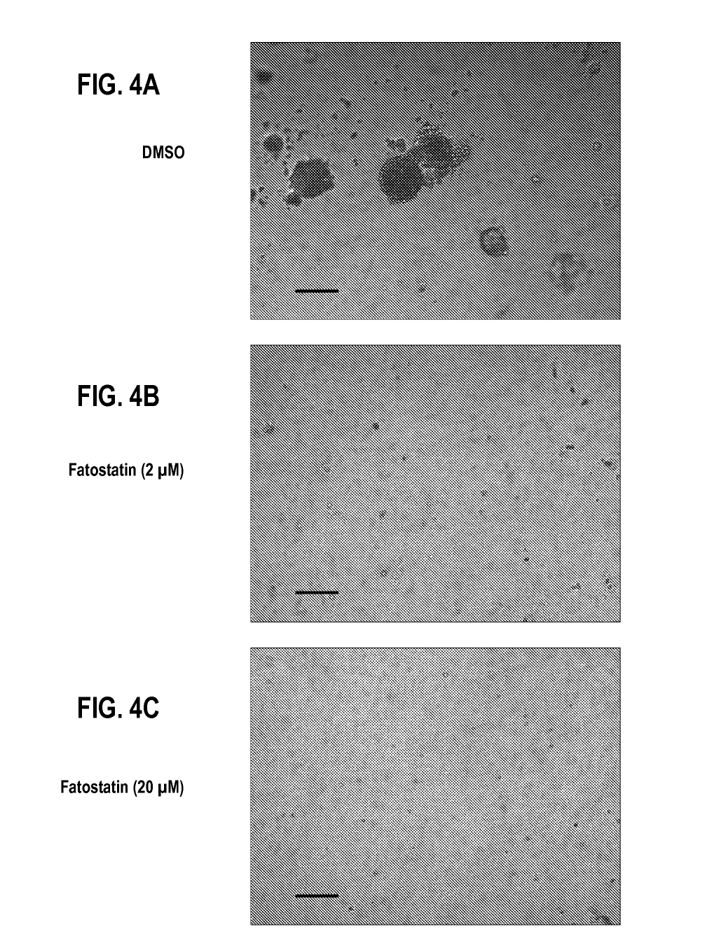

It has been discovered that fatostatin treatment had a dramatic effect on normalizing the abnormal 3D morphology of 3 strains of breast cancer cells: MDA-468 cells (FIGS. 1 and 4), MDA-231 cells (FIG. 2), and SKBR3 cells (FIG. 3). The results prove a functional interaction with SREBPs as being critical for mutant p53-mediated upregulation of the mevalonate pathway genes. The following is a summary of results of experiments described in the Examples of this application.

Fatostatin normalized abnormal cell morphology in p53 breast cancer cell lines;

Fatostatin inhibited MDA-231 cell growth in 3D culture;

Fatostatin inhibited MDA-468 cell growth in 3D culture; and

Fatostatin inhibited SKBR3 cell growth in 3D culture.

### Methods for Detecting p53 Mutations

The subgroup of breast cancer patients displaying p53 mutations generally respond poorly to therapy and exhibit rapidly growing tumors and shorter median survival (Lai et al., supra; Reed (1996) J. Clin. Invest., 97:2403-2404). Aberrant forms of human p53 are associated with poor prognosis, more aggressive tumors, metastasis, and short survival rates (Mitsudomi et al., Clin Cancer Res 2000 October; 6(10):4055-63; Koshland, Science (1993) 262:1953). The Gene ID for TP53 is 7157.

Alterations or mutations of a wild-type p53 gene according to the present invention encompass all forms of mutations such as insertions, inversions, deletions, and/or point mutations. Somatic mutations are those which occur only in certain tissues, e.g., in the tumor tissue, and are not inherited in the germ line. If only a single allele is somatically mutated, an early neoplastic state is indicated. However, if both alleles are mutated then a late neoplastic state is indicated. Germ line mutations can be found in any of a body's tissues. Patients who have Li-Fraumeni inherit germ-line mutations in TP53, however germ line TP53 mutations are rare. In an embodiment Li-Fraumeni patients can be treated by administering a therapeutic agent that inhibits one or more enzymes in the mevalonate pathway to treat or prevent cancer that has a p53mutation. The finding of p53 mutations in a benign tumor is also a condition that can be treated prophylactically.

Cancer (and precancerous lesions or benign tumors) that express a mutant p53 gene or a mutant form of p53 protein or an mRNA encoding a mutant form of p53 protein can be treated or prevented with the methods of the present invention. Such cancers include breast cancer, neuroblastoma, gastrointestinal carcinoma such as rectum carcinoma, colon carcinoma, familial adenomatous polyposis carcinoma and hereditary non-polyposis colorectal cancer, esophageal carcinoma, labial carcinoma, laryngeal carcinoma, hypopharyngial carcinoma, tongue carcinoma, salivary gland carcinoma, gastric carcinoma, medullary thyroid carcinoma, papillary thyroid carcinoma, renal carcinoma, kidney parenchymal carcinoma, ovarian carcinoma, cervical carcinoma, uterine corpus carcinoma, endometrium carcinoma, choriocarcinoma, pancreatic carcinoma, prostate carcinoma, testis carcinoma, urinary carcinoma, melanoma, brain tumors such as glioblastoma, astrocytoma, meningioma, medulloblastoma and peripheral neuroectodermal tumors, Hodgkin's lymphoma, non-Hodgkin's lymphoma, Burkitt's lymphoma, acute lymphocytic leukemia (ALL), chronic lymphocytic leukemia (CLL), acute myelologenous leukemia (AML), chronic myelologenous leukemia (CML), adult T-cell leukemia/lymphoma, hepatocellular carcinoma, gallbladder carcinoma, bronchial carcinoma, small cell lung carcinoma, non-small cell lung carcinoma, multiple myeloma, basal cell carcinoma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing's sarcoma and plasmocytoma. Particular tumors include those of the brain, liver, kidney, bladder, breast, gastric, ovarian, colorectal, prostate, pancreatic, lung, vulval, thyroid, colorectal, oesophageal, sarcomas, glioblastomas, head and neck, leukemias and lymphoid malignancies.

Mutant p53 genes or gene products can be detected in tumor samples or, in some types of cancer, in biological samples such as urine, stool, sputum or serum. For example, TP53 mutations can often be detected in urine for bladder cancer and prostate cancer, sputum for lung cancer, or stool for colorectal cancer. Serum has mostly been tested in the context of colorectal cancer, however this should work for any tumor type that sheds cancer cells into the blood. Cancer cells are found in blood and serum for cancers such as lymphoma or leukemia. The same techniques discussed above for detection of mutant p53 genes or gene products in tumor samples can be applied to other body samples. Cancer cells are sloughed off from tumors and appear in such body samples.

A p53 (TP53) gene mutation in a sample can be identified using any method known in the art. Examples of TP53 mutations are described in, e.g., Soussi T. (2007) Cancer Cell 12(4):303-12; Cheung K. J. (2009) Br J. Haematol. 146(3):257-69; Pfeifer G. P. et al. (2009) Hum Genet. 125(5-6):493-506; Petitjean A. et al. (2007) Oncogene 26(15):2157-65. One of the most commonly used methods to “identify” p53 mutants is by utilizing immunohistochemistry (IHC) on tumor sections stained with a p53 antibody. Positive staining with an antibody against p53 is often used as a surrogate for sequencing the gene itself. Some have proposed combining sequencing and IHC, since p53 mutants that are highly expressed tend to be more oncogenic.

In one assay, nucleic acid from the sample is contacted with a nucleic acid probe that is capable of specifically hybridizing to nucleic acid encoding a mutated p53 protein, or fragment thereof incorporating a mutation, and detecting the hybridization. In a particular embodiment the probe is detectably labeled such as with a radioisotope, a fluorescent agent (rhodamine, fluorescene) or a chromogenic agent. In a particular embodiment the probe is an antisense oligomer. The probe may be from about 8 nucleotides to about 100 nucleotides, or about 10 to about 75, or about 15 to about 50, or about 20 to about 30. Kits for identifying p53 mutations in a sample are available that include an oligonucleotide that specifically hybridizes to or adjacent to a site of mutation in the p53 gene. The p53 Amplichip™ developed by Roche is a good example of this technology.

Using gene expression signatures, it has been shown that most p53 mutations cluster in the basal-like subgroup of breast cancers, which has the poorest prognosis and is notoriously difficult to treat (Perou et al., 2000). Using a combination of expression signatures and data from over 40,000 compounds screened in the NCI-60 cell lines, Mori et al. predicted three FDA-approved drugs to be most effective for treating basal-like breast cancers, two of which, Simvastatin and Lovastatin, are inhibitors of HMG-CoA reductase (Mori et al., 2009). Embodiments of the present invention provide a means for stratifying breast cancer patients based on their p53 mutational status to identify patients who will respond to treatment with a statin or other inhibitor of one or more enzymes in the mevalonate pathway.

Not all p53 mutations are equivalent. Genetic alterations in p53 are often grouped into two classes based on the type of mutant p53 that they produce (Brosh and Rotter, 2009). Contact mutants, exemplified by p53-R273H, involve mutation of residues that are directly involved in protein-DNA contacts. Conformational mutants, typified by p53-R175H, result in conformational distortions in the p53 protein. The experimental results herein show that a subset of the sterol biosynthesis genes are significantly higher in large cohorts of human breast tumors bearing mutant p53 which shows that the ability of mutant p53 to upregulate the sterol biosynthesis genes is not constrained to a single class of p53 mutations. Thus the present methods for reducing or eliminating treating cancer, precancerous lesions or preventing benign tumors with p53 mutations from becoming cancerous can be broadly used for any p53 mutation.

A mutation in the p53 gene in a sample can be detected by amplifying nucleic acid corresponding to the p53 gene obtained from the sample, or a biologically active fragment, and comparing the electrophoretic mobility of the amplified nucleic acid to the electrophoretic mobility of corresponding wild-type p53 gene or fragment thereof. A difference in the mobility indicates the presence of a mutation in the amplified nucleic acid sequence. Electrophoretic mobility may be determined on polyacrylamide gel. Alternatively, an amplified p53 gene or fragment nucleic acid may be analyzed for detection of mutations using Enzymatic Mutation Detection (EMD) (Del Tito et al, Clinical Chemistry 44:731-739, 1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel electrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

In order to detect the mutation of the wild-type p53 gene, a sample or biopsy of the tumor or a sample comprising cancer cells or precancerous cells (such as blood, serum, CSF, stool, urine or sputum) is obtained by methods well known in the art and appropriate for the particular type and location of the tumor. For instance, samples of breast cancer lesions may be obtained by resection, or fine needle aspiration. Means for enriching a tissue preparation for tumor cells are known in the art. For example, the tissue may be isolated from paraffin or cryostat sections. Cancer cells may also be separated from normal cells by flow cytometry or laser capture microdissection. These as well as other techniques for separating tumor from normal cells are well known in the art. If the tumor tissue is highly contaminated with normal cells, detection of mutations is more difficult.

Detection of point mutations may be accomplished by molecular cloning of the p53 allele (or alleles) and sequencing that allele(s) using techniques well known in the art. Alternatively, the polymerase chain reaction (PCR) can be used to amplify gene sequences directly from a genomic DNA preparation from the tumor tissue. The DNA sequence of the amplified sequences can then be determined and mutations identified. The polymerase chain reaction is the preferred method and it is well known in the art and described in Saiki et al., Science 239:487, 1988; U.S. Pat. Nos. 4,683,203; and 4,683,195.

The ligase chain reaction, which is known in the art, can also be used to amplify p53 sequences. See Wu et al., Genomics, Vol. 4, pp. 560-569 (1989). In addition, a technique known as allele specific PCR can be used. (See Ruano and Kidd, Nucleic Acids Research, Vol. 17, p. 8392, 1989.) According to this technique, primers are used which hybridize at their 3′ ends to a particular p53 mutation. If the particular p53 mutation is not present, an amplification product is not observed. Amplification Refractory Mutation System (ARMS) can also be used as disclosed in European Patent Application Publication No. 0332435 and in Newton et al., Nucleic Acids Research, Vol. 17, p. 7, 1989. Insertions and deletions of genes can also be detected by cloning, sequencing and amplification. In addition, restriction fragment length polymorphism, (RFLP) probes for the gene or surrounding marker genes can be used to score alteration of an allele or an insertion in a polymorphic fragment. Single stranded conformation polymorphism (SSCP) analysis can also be used to detect base change variants of an allele. (Orita et al., Proc. Natl. Acad. Sci. USA Vol. 86, pp. 2766-2770, 1989, and Genomics, Vol. 5, pp. 874-879, 1989.) Other techniques for detecting insertions and deletions as are known in the art can be used.

Mismatches, according to the present invention are hybridized nucleic acid duplexes which are not 100% complementary. The lack of total complementarity may be due to deletions, insertions, inversions, substitutions or frameshift mutations. Mismatch detection can be used to detect point mutations in the gene or its mRNA product. While these techniques are less sensitive than sequencing, they are simpler to perform on a large number of tumor samples. An example of a mismatch cleavage technique is the RNase protection method, which is described in detail in Winter et al., Proc. Natl. Acad. Sci. USA, Vol. 82, p. 7575, 1985 and Meyers et al., Science, Vol. 230, p. 1242, 1985. A labeled riboprobe which is complementary to the human wild-type p53 gene coding sequence can also be used. The riboprobe and either mRNA or DNA isolated from the tumor tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the p53 mRNA or gene. If the riboprobe comprises only a segment of the p53 mRNA or gene it will be desirable to use a number of these probes to screen the whole mRNA sequence for mismatches.

In a similar manner, DNA probes can be used to detect mismatches, through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the cellular mRNA or DNA which might contain a mutation can be amplified using PCR before hybridization. Changes in DNA of the p53 gene can also be detected using Southern hybridization, especially if the changes are gross rearrangements, such as deletions and insertions.

DNA sequences of the p53 gene which have been amplified by use of polymerase chain reaction may also be screened using allele-specific probes. These probes include nucleic acid oligomers, each of which contains a region of the p53 gene sequence harboring a known mutation. For example, one oligomer may be about 30 nucleotides in length, corresponding to a portion of the p53 gene sequence. By use of a battery of such allele-specific probes, PCR amplification products can be screened to identify the presence of a previously identified mutation in the p53 gene. Hybridization of allele-specific probes with amplified p53 sequences can be performed, for example, on a nylon filter. Hybridization to a particular probe under stringent hybridization conditions indicates the presence of the same mutation in the tumor tissue as in the allele-specific probe. This is used with the p53 Amplichip described above.

Alteration of wild-type p53 genes can also be detected by screening for alteration of wild-type p53 protein. For example, monoclonal antibodies immunoreactive with p53 can be used to screen a tissue. As mentioned above, one of the common ways to “detect” p53 mutations is to see strong p53 immunostaining in tissue sections (these are not mutant p53 specific antibodies, but simply take advantage of the fact that most mutant p53 proteins are more stable (and thus more abundant) than wild-type p53. Antibodies specific for products of mutant alleles could also be used to detect mutant p53 gene product. Such immunological assays can be done in any convenient format known in the art. These include Western blots, immunohistochemical assays and ELISA assays. Any means for detecting an altered p53 protein or p53 mRNA can be used to detect alteration of wild-type p53 genes or the expression product of the gene. Point mutations may be detected by amplifying and sequencing the mRNA or via molecular cloning of cDNA made from the mRNA (or by sequencing genomic DNA). The sequence of the cloned cDNA can be determined using DNA sequencing techniques which are well known in the art. The cDNA can also be sequenced via the polymerase chain reaction (PCR).

### Methods of Treatment

Certain embodiments of the invention provide methods for treating or preventing cancer, or for reducing or eliminating precancerous cells, or a benign tumor in a subject that have a mutated p53 gene or that express a mutant p53 protein or an mRNA encoding a mutant p53 protein, by administering to the subject a therapeutically or prophylactically effective amount of an SREBP cleavage activating protein inhibitor, such as fatostatin or an analogue thereof. To identify subjects that will respond to treatment a biological sample of the cancer, the precancerous cells or the benign tumor is obtained from the subject. If it is determined that the cancer cells, the precancerous cells, or the cells of the benign tumor in the biological sample have the mutant p53 gene or express the mutant p53 protein or a mRNA encoding the mutant p53 protein; then the subject will respond to treatment with the SREBP cleavage activating protein inhibitor. Biological samples in certain embodiments include, but are not limited to, tumor biopsies, urine, blood, cerebrospinal fluid, sputum, serum, stool, or bone marrow.

As is described above, statins are known to be effective in treating cancers with p53 mutations. Therefore certain other embodiments are directed to combination therapy for reducing or eliminating cancer, precancerous lesions or benign tumors with both fatostatin (or fatostatin analogue) and one or more statins. The drugs can be administered at the same time or at different times. They can be administered orally, by injection, parenterally, by inhalation spray, topically, rectally, nasally, buccally, vaginally, or via an implanted reservoir. Fatostatin can be administered locally to the site of the cancer or benign tumor. Another embodiment is directed to a pharmaceutical formulation comprising fatostatin and one or more statins selected from the group comprising rosuvastatin, lovastatin, simvastatin, pravastatin, rosuvastatin, fluvastatin, atorvastatin, and cerivastatin.

Certain embodiments of the present invention are directed to methods for determining if a subject with cancer or precancerous lesions or a benign tumor, will respond to treatment (i.e. if the patient and the cancer will respond to treatment) with a SREBP cleavage activating protein inhibitor such as fatostatin or an analogue thereof by (i) obtaining a sample of the cancer cells, the precancerous cells or the benign tumor cells from the subject, (ii) assaying the cells in the sample for the presence of a mutated p53 gene or a mutant form of p53 protein or a biologically active fragment thereof or an mRNA encoding a mutant p53 protein, and (iii) if detected, then determining that the subject will respond to treatment with the inhibitor or combinations. Yet other embodiments are directed to a method of preventing recurrence of cancer, precancerous lesions or a benign tumor or methods of preventing cancer in a subject at high risk of developing cancer comprising a p53 protein or gene mutation or mRNA encoding mutant p53 protein, by administering fatostatin or an analogue thereof, alone or together as a combination treatment with a statin.

### Administration of Therapeutic Agents

As defined herein, a “therapeutic agent” is an SREBP cleavage activating protein inhibitor, including fatostatin or fatostatin analogues such as, but not limited to, 4-(2-Methoxyphenyl)-2-(2-propylpyridin-4-yl)thiazole; N-Isopropyl-4-(2-(2-propylpyridin-4-yl)thiazol-4-yl)aniline; and N-(4-(2-(2-Propylpyridin-4-yl)thiazol-4-yl)phenyl)methanesulfonamide (as described in Kamisuki, Shinji et al. (2011). The therapeutically effective amount of a therapeutic agent depends upon a number of factors within the ordinarily skill of a physician, veterinarian, or researcher and will vary depending inter alia on the subject, the activity and bioavailability of the specific agent(s) employed, the age, body weight, general health, gender, and diet of the subject, the time of administration, the route of administration, the rate of excretion, and the drug itself or combination of drugs. Contributing factors further include the type, location, aggressiveness and size of cancer, precancerous lesion or benign tumor. Some highly aggressive tumors may require higher therapeutic amounts, for example. The full therapeutic effect does not necessarily occur by administration of one dose of the agent and may occur only after administration of a series of doses. Thus, a therapeutically effective amount may be administered in one or more administrations, on the same day or on different days.

The therapeutic agent such as fatostatin or an analogue thereof may be administered alone or in combination with a statin. All statins block the same enzyme HMGCoA reductase and they have same binding site and mechanism of action. However, they have different bioavailability and tissue specificity. In an embodiment, formulations of statins for treating brain cancer or reducing precancerous lesions or benign tumors in the brain or central nervous system comprise one or more lipophilic statins in a therapeutically effective amount.

In the in vitro experiments described herein, amounts of fatostatin ranged from 2 μM-μM and were shown to have dramatic effects on 3D morphology of breast cancer cell lines.

In the in vivo experiments described by Kamisuki, Shinji et al., (2009) and Kamisuki, Shinji et al., (2011) using mice, fatostatin was administered via i.p. injection at a dose of 30 mg/kg/; 150 μL) to determine effects of fatostatin on body weight, blood constitutents, and liver and adipose tissues. Fatostatin analogues for use in the present invention can be synthesized and tested as described in Kamisuki, Shinji et al. (2011). Oral availability was demonstrated in mice after administration of 23 mg/kg of the fatostatin analogue N-(4-(2-(2-Propylpyridin-4-yl)thiazol-4-yl)phenyl)methanesulfonamide.

Suggested therapeutically effective amounts of fatostatin or fatostatin analogue for use in various embodiments of the present invention for administration to humans range from about 0.1 mg/kg to about 150 mg/kg to treat cancer, or to reduce or eliminate precancerous cells or a benign tumor that has a mutated p53 gene or that expresses a mutant p53 protein or an mRNA encoding a mutant p53 protein. A person of skill in the art can determine the therapeutically effective amount of fatostatin. Factors affecting the dose include the aggressiveness of the cancer, the route of administration, the frequency of administration, bioavailability of the drug, the health of the subject, and whether the condition is treatment of a precancerous condition or a benign tumor.

Therapeutic agents may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraruterial, subcutaneous, intraperitoneal or intramuscular injection or infusion; or intracranial, e.g., intrathecal or intraventricular, administration. In some embodiments a slow release preparation comprising the therapeutic agents is administered. The therapeutic agents can be administered as a single treatment or in a series of treatments that continue as needed and for duration of time that causes one or more symptoms of the cancer to be reduced or ameliorated, or that achieves another desired effect.

The dose(s) vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

A suitable subject is an individual or animal that has cancer, a precancerous lesion or has a benign tumor that has a p53 mutation, or expresses mutant p53 protein or an mRNA encoding mutant p53 protein. Administration of a therapeutic agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for administration).

### Pharmaceutical Compositions or Formulations

An embodiment is directed to a pharmaceutical composition comprising therapeutically effective amounts of fatostatin or a fatostatin analogue (as described in Kamisuki, Shinji et al., 2011) in a range of from about 0.1 mg/kg to about 150 mg/kg, that can be optionally formulated to further include one or more statins in therapeutically effective amounts ranging from below 80 mg up to 1 gm. The therapeutic agents may be present in the pharmaceutical compositions in the form of salts of pharmaceutically acceptable acids or in the form of bases. The therapeutic agents may be present in amorphous form or in crystalline forms, including hydrates and solvates. Preferably, the pharmaceutical compositions comprise a therapeutically effective amount.

Pharmaceutically acceptable salts of the therapeutic agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining phamlaceutically acceptable acid addition salts.

Salts derived from appropriate bases include alkali metal (e.g., sodium and potassium), alkaline earth metal (e.g., magnesium), ammonium and salts. This invention also envisions the qmitemization of any basic nitrogen-containing groups of the therapeutic agents disclosed herein. Water or oil-soluble or dispersible products may be obtained by such quaternization.

The therapeutic agents of the present invention are also meant to include all stereochemical forms of the therapeutic agents (i.e., the R and S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of the present invention are also meant to include compounds which differ only in the presence of one or more isotopically enriched atoms. For example, therapeutic agents in which one or more hydrogens are replaced by deuterium or tritium, or the replacement of one or more carbons by 13C- or 14C-enriched carbon are within the scope of this invention.

In a preferred embodiment, the therapeutic agents of the present invention are administered in a pharmaceutical composition that includes a pharmaceutically acceptable carrier, adjuvant, or vehicle. The term “pharmaceutically acceptable carrier, adjuvant, or vehicle” refers to a non-toxic carrier, adjuvant, or vehicle that does not destroy or significantly diminish the pharmacological activity of the therapeutic agent with which it is formulated. Pharmaceutically acceptable carriers, adjuvants or vehicles that may be used in the compositions of this invention encompass any of the standard pharmaceutically accepted liquid carriers, such as a phosphate-buffered saline solution, water, as well as emulsions such as an oil/water emulsion or a triglyceride emulsion. Solid carriers may include excipients such as starch, milk, sugar, certain types of clay, stearic acid, talc, gums, glycols, or other known excipients. Carriers may also include flavor and color additives or other ingredients. The formulations of the combination of the present invention may be prepared by methods well-known in the pharmaceutical arts and described herein. Exemplary acceptable pharmaceutical earners have been discussed above. An additional carrier, Cremophor™, may be useful, as it is a common vehicle for Taxol.

The pharmaceutical compositions of the present invention are preferably administered orally, preferably as solid compositions. However, the pharmaceutical compositions may be administered parenterally, by inhalation spray, topically, rectally, nasally, buccally, vaginally or via an implanted reservoir. Sterile injectable forms of the pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The pharmaceutical compositions employed in the present invention may be orally administered in any orally acceptable dosage form, including, but not limited to, solid forms such as capsules and tablets. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions employed in the present invention may also be administered by nasal aerosol or inhalation. Such pharmaceutical compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier. The passage of the agent can also be facilitated by its covalent coupling to a peptide vector capable of transporting the agent through the blood-brain barrier. Peptide transport vectors known as blood-brain barrier permeabilizer compounds are disclosed in U.S. Pat. No. 5,268,164. Site specific macromolecules with lipophilic characteristics useful for delivery to the brain are disclosed in U.S. Pat. No. 6,005,004.

Examples of routes of administration comprise parenteral, e.g., intravenous, intradermal, subcutaneous, inhalation, transdermal (topical), transmucosal, and rectal administration; or oral. Solutions or suspensions used for parenteral, intradermal, or subcutaneous application can comprise the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injection comprise sterile aqueous solutions (where water soluble) or dispersions and sterile powders for the extemporaneous preparation of sterile injectable solutions or dispersions. For intravenous administration, suitable carriers comprise physiological saline, bacteriostatic water, Cremophor EL™ (BASF, Parsippany, N. J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as manitol, sorbitol, or sodium chloride. Prolonged absorption of an injectable composition can be achieved by including in the composition an agent that delays absorption, for example, aluminum monostearate or gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle that contains a basic dispersion medium and other ingredients selected from those enumerated above or others known in the art. In the case of sterile powders for the preparation of sterile injectable solutions, the methods of preparation comprise vacuum drying and freeze-drying which yields a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be comprised as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Ptimogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be pemleated are used in the formulation. Such penetrants are generally known in the art, and comprise, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### EXAMPLES

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### Materials and Methods

### Animals

Fatostatin was synthesized by the Medicinal Chemistry Core Facility at the Sanford-Burnham Medical Research Institute as previously described (Kamisuki et al., 2009).

### Cell Lines and Generation of Stable Cell Lines

MDA-468 and MDA-231 cells were maintained in DMEM+10% FBS. SKBR3 cells were maintained in McCoy's 5a medium+10% FBS. All cells were maintained at 37° C. in 5% CO₂. To generate stable cell lines with inducible shRNA, constructs were introduced into MDA-231 or MDA-468 cells by the retroviral mediated gene transfer method. The generated viruses were harvested and MDA-231 or MDA-468 cells were co-infected with the rtTA and one of the vectors. After selection with puromycin (vector with shRNA) and hygromycin (rtTA), clonal cell lines were generated by the limited dilution method. Clonal cell lines were selected based on the extent of p53 knockdown. Experiments were carried out on clonal cell lines or stable pools (MDA-468.shp53 pool, MDA-468.shp53 clone 1F5 and MDA-231.shp53 clone 1D10).

### Three-Dimensional Culture

Three-dimensional culture was carried out as described in Debnath et al., 2003. Briefly, 8-well chamber slides were lined with 50 μl growth factor reduced Matrigel (BD Biosciences). Cells were then seeded at a density of 5,000 cells/well in Assay Medium (DMEM/F12+2% Horse Serum+10 μg/ml Insulin+0.5 μg/ml Hydrocortisone) containing 2% Matrigel. Cells were refed with Assay Medium containing 2% Matrigel every 4 days. For RNA/protein analysis from 3D cultures, 35 mm plates were lined with 500 μl Matrigel and cells were seeded at a density of 225,000 cells/plate in Assay Medium+2% Matrigel. Cells were harvested using Cell Recovery Solution (BD Biosciences) according to the manufacturer's instructions.

### Quantitative Chromatin Immunoprecipitation

Chromatin Immunoprecipitation (ChIP) experiments were carried out as described in Beckerman et al., 2009. Briefly, MDA-468 cells were treated with 1% formaldehyde prior to lysis in RIPA Buffer and sonication to yield 500 bp fragments. Protein A/G Sepharose beads were conjugated to anti-p53 antibodies (1801/D0-1) which were subsequently used to immunoprecipitate p53 from 1 mg whole cell lysate. Quantitative ChIP was carried out on an ABI StepOne Plus using SYBR green dye. Genomic Locations of SRE-1 sites within the promoters of sterol biosynthesis genes were located using a literature search: HMGCS 1 (Inoue et al., 1998), HMGCR (Boone et al., 2009), MVK (Bishop et al., 1998), FDPS (Ishimoto et al., 2010), FDFT1 (Inoue et al., 1998), SQLE (Nagai et al., 2002) and CYP51A1 (Halder et al., 2002), respectively. ChIP primer sequences are provided in Table 2 of PCT/US 11/55488 and are incorporated by reference.

### Example 2

### MDA-468.shp53 Cells Treated with Fatostatin

MDA-468.shp53 cells were treated with Fatostatin (20 μM) and subjected to ChIP analysis. FIG. 1. Cells in 3D culture were treated on Day 1 or Day 4 of the 3D protocol and refed every 4 days with fresh drug. Data are presented as mean+−SD of six independent experiments. **p<0.01.

### Example 3

### MDA-231.shp53 Cells Treated with Fatostatin

MDA-231.shp53 cells were grown in 3D cultures for 8 days and treated with DMSO and fatostatin (2 or 20 μM). Drugs were added on day 1. Representative DIC images are shown. FIG. 2. Scale bar, 200 p.m.

### Example 4

### Fatostatin Inhibits SKBR3Cell Growth in 3D Culture

SKBR3 cells were grown in 3D cultures for 8 days treated with DMSO, Fatostatin (2 μM) or (20 μM). Drugs were added on Day 1. Representative Differential Interference Contrast (DIC) images are shown in FIG. 3.

### Example 5

### Fatostatin Inhibits MDA-468 Cell Growth in 3D Culture

MDA-468.shp53 cells were grown in 3D cultures for 10 days treated with DMSO, Fatostatin (2 μM) or (20 μM). Drugs were added on Day 1. Representative Differential Interference Contrast (DIC) images are shown in FIG. 4.

### Example 6

### Prophetic Mouse Study

MDA-231 cells (2×10⁶), resuspended in 50 μl media+50 μl Matrigel, will be injected subcutaneously into 8 week-old female NOD-SCID mice. 14 days after implantation, tumors will be measured by calipers and mice will be paired by equal tumor volume and randomized to a Fatostatin or Control group (N=5 in both cases). On day 1 of the experiment, and every day thereafter, the weight and the amount of food intake of each mouse will be measured. The fatostatin mice will then receive an i.p. injection of fatostatin (of about 30 mg/kg; 150 μL) or oral dosage of a fatostatin analogue (23 mg/kg) while the control mice receive 10% DMSO in PBS. Daily injections will be continued for 28 days, when the study is ended. Each mouse on the standard chow dies receives between 0.75 mg (25 g mouse) to 0.9 mg (30 g mouse) of fatostatin or a fatostatin analogue depending on body weight. Mice will be maintained at a 25-30 g body weight on normal chow diet (11% fat). Mice will be weighed weekly and tumor measurements will be performed weekly using a caliper. The volume of the tumor may be calculated as v=a2*b (a being the small diameter and b the long diameter). After 28 days of treatment, mice can be sacrificed and tumors will be extracted and weighed. These experiments can be repeated to determine the optimum effective dose of therapeutic agent.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application (including the Appendix and reference lists) are hereby expressly incorporated by reference as if set forth herein in their entirety, except where terminology is not consistent with the definitions herein. Although specific terms are employed, they are used as in the art unless otherwise indicated.

### REFERENCES

Adorno, M., Cordenonsi, M., Montagner, M., Dupont, S., Wong, C., Hann, B., Solari, A., Bobisse, S., Rondina, M. B., Guzzardo, V., et al. (2009). A Mutant-p53/Smad complex opposes p63 to empower TGFbeta-induced metastasis. Cell 137, 87-98.
Ahern, T. P., Pedersen, L., Tarp, M., Cronin-Fenton, D. P., Game, J. P., Silliman, R. A., Sorensen, H. T., and Lash, T. L. (2011). Statin Prescriptions and Breast Cancer Recurrence Risk: A Danish Nationwide Prospective Cohort Study. J Natl Cancer Inst.
Anderson; et al. General nucleic acid hybridization methods. (1999) Nucleic Acid Hybridization, BIOS Scientific Publishers.
Ausubel et al., (1999) Applicable PCR amplification techniques are described in Current Protocols in Molecular Biology, John Wiley & Sons, Inc., New York.
Baigent, C., Keech, A., Kearney, P. M., Blackwell, L., Buck, G., Pollicino, C., Kirby, A., Sourjina, T., Peto, R., Collins, R., et al. (2005). Efficacy and safety of cholesterol-lowering treatment: prospective meta-analysis of data from 90,056 participants in 14 randomised trials of statins. Lancet 366, 1267-1278.
Baldi et al., (2003) Microarrays Methods and Applications: Nuts & Bolts. DNA Press.
Baldi et al., (2002) DNA Microarrays and Gene Expression: From Experiments to Data Analysis and Modeling, Cambridge University Press.
Barsotti, A. M., and Prives, C. (2009). Pro-proliferative FoxMl is a target of p53-mediated repression. Oncogene 28, 4295-4305.
Beckerman, R., Donner, A. J., Mattia, M., Peart, M. J., Manley, J. L., Espinosa, J. M., and Prives, C. (2009). A role for Chk1 in blocking transcriptional elongation of p21 RNA during the S-phase checkpoint. Genes Dev 23, 1364-1377.
Bissell, M. J., Kenny, P. A., and Radisky, D. C. (2005). Microenvironmental regulators of tissue structure and function also regulate tumor induction and progression: the role of extracellular matrix and its degrading enzymes. Cold Spring Harb Symp Quant Biol 70, 343-356.
Bissell, M. J., Radisky, D. C., Rizki, A., Weaver, V. M., and Petersen, O. W. (2002). The organizing principle: microenvironmental influences in the normal and malignant breast. Differentiation 70, 537-546.
Blais, L., Desgagne, A., and LeLorier, J. (2000). 3-Hydroxy-3-methylglutaryl coenzyme A reductase inhibitors and the risk of cancer: a nested case-control study. Arch Intern Med 160, 2363-2368.
Bossi, G., Marampon, F., Maor-Aloni, R., Zani, B., Rotter, V., Oren, M., Strano, S., Blandino, G., and Sacchi, A. (2008). Conditional RNA interference in vivo to study mutant p53 oncogenic gain of function on tumor malignancy. Cell Cycle 7, 1870-1879.
Brosh, R., and Rotter, V. (2009). When mutants gain new powers: news from the mutant p53 field. Nat Rev Cancer 9, 701-713.
Browning, D. R., and Martin, R. M. (2007). Statins and risk of cancer: a systematic review and metaanalysis. Int J Cancer 120, 833-843.
Buchwald, H. (1992). Cholesterol inhibition, cancer, and chemotherapy. Lancet 339, 1154-1156.
Campbell, M. J., Esserman, L. J., Zhou, Y., Shoemaker, M., Lobo, M., Borman, E., Baehner, F., Kumar, A. S., Adduci, K., Marx, C., et al. (2006). Breast cancer growth prevention by statins. Cancer Res 66, 8707-8714.
Cariello, et al., (1988) Human Genetics, Vol. 42, p. 726,
Cauley, J. A., Zmuda, J. M., Lui, L. Y., Hillier, T. A., Ness, R. B., Stone, K. L., Cummings, S. R., and Bauer, D. C. (2003). Lipid-lowering drug use and breast cancer in older women: a prospective study. J Womens Health (Larchmt) 12, 749-756.
Clendening, J. W., Pandyra, A., Boutros, P. C., El Ghamrasni, S., Khosravi, F., Trentin, G. A., Martirosyan, A., Hakem, A., Hakem, R., Jurisica, I., et al. (2010). Dysregulation of the mevalonate pathway promotes transformation. Proc Natl Acad Sci USA 107, 15051-15056.
Cotton et al., Proc. Natl. Acad. Sci. USA, Vol. 85, 4397, 1988; and Shenk et al., Proc. Natl. Acad. Sci. USA, Vol. 72, p. 989, 1975
Debnath, J., Muthuswamy, S. K., and Brugge, J. S. (2003). Morphogenesis and oncogenesis of MCF-10A mammary epithelial acini grown in three-dimensional basement membrane cultures. Methods 30, 256-268.
Garwood, E. R., Kumar, A. S., Baehner, F. L., Moore, D. H., Au, A., Hylton, N., Flowers, C. I., Garber, J., Lesnikoski, B. A., Hwang, E. S., et al. (2010). Fluvastatin reduces proliferation and increases apoptosis in women with high grade breast cancer. Breast Cancer Res Treat 119, 137-144.
Ghosh-Choudhury, N., Mandal, C. C., and Ghosh Choudhury, G. (2010). Simvastatin induces derepression of PTEN expression via NFkappaB to inhibit breast cancer cell growth. Cell Signal 22, 749-758.
Goldstein, J. L., and Brown, M. S. (1990). Regulation of the mevalonate pathway. Nature 343, 425-430.
Graaf, M. R., Richel, D. J., van Noorden, C. J., and Guchelaar, H. J. (2004). Effects of statins and farnesyltransferase inhibitors on the development and progression of cancer. Cancer Treat Rev 30, 609-641.
Horton, J. D., Goldstein, J. L., and Brown, M. S. (2002). SREBPs: activators of the complete program of cholesterol and fatty acid synthesis in the liver. J Clin Invest 109, 1125-1131.
Ignaszak-Szczepaniak et al., (2006) Oncol Rep 16:65-7
Innis et al., (1999) PCR Applications: Protocols for Functional Genomics, 1st Edition, Academic Press.
Kamisuki, Shinji et al., (2009) A Small Molecule That Blocks Fat Synthesis By Inhibiting the Activation of SREBP, in Chemistry & Biology 16, 882-892.
Kamisuki, Shinji et al., (2011) Synthesis and Evaluation of Diarylthiazole Derivatives That Inhibit Activation of Sterol Regulatory Element-Binding Proteins, J. Med. Chem., 54, 4923-4927.
Kamisuki, S., Mao, Q., Abu-Elheiga, L., Gu, Z., Kugimiya, A., Kwon, Y., Shinohara, T., Kawazoe, Y., Sato, S., Asakura, K., et al. (2009). A small molecule that blocks fat synthesis by inhibiting the activation of SREBP. Chem Biol 16, 882-892.
Kenny, P. A., Lee, G. Y., Myers, C. A., Neve, R. M., Semeiks, J. R., Spellman, P. T., Lorenz, K., Lee, E. H., Barcellos-Hoff, M. H., Petersen, O. W., et al. (2007). The morphologies of breast cancer cell lines in three-dimensional assays correlate with their profiles of gene expression. Mol Oncol 1, 84-96.
Kidera, Y., Tsubaki, M., Yamazoe, Y., Shoji, K., Nakamura, H., Ogaki, M., Satou, T., Itoh, T., Isozaki, M., Kaneko, J., et al. (2010). Reduction of lung metastasis, cell invasion, and adhesion in mouse melanoma by statin-induced blockade of the Rho/Rho-associated coiled-coil-containing protein kinase pathway. J Exp Clin Cancer Res 29, 127.
Kim, E., Giese, A., and Deppert, W. (2009). Wild-type p53 in cancer cells: when a guardian turns into a blackguard. Biochem Pharmacol 77, 11-20.
Koyuturk, M., Ersoz, M., and Altiok, N. (2007). Simvastatin induces apoptosis in human breast cancer cells: p53 and estrogen receptor independent pathway requiring signalling through JNK. Cancer Lett 250, 220-228.
Krepinsky et al., (2011) Articles in Pres S. Am J Physiol Renal Physiol. doi:10.1152/ajprenal.00136.2011; SREBP-1 Activation by Glucose Mediates TGFβ Upregulation in Mesangial Cells;
Kubatka, P., Zihlavnikova, K., Kajo, K., Pec, M., Stollarova, N., Bojkova, B., Kassayova, M., and Orendas, P. (2011). Antineoplastic effects of simvastatin in experimental breast cancer. Klin Onkol 24, 41-45.
Kumar, A. S., Benz, C. C., Shim, V., Minami, C. A., Moore, D. H., and Esserman, L. J. (2008). Estrogen receptor-negative breast cancer is less likely to arise among lipophilic statin users. Cancer Epidemiol Biomarkers Prey 17, 1028-1033.
Lai et al. (2004) Breast Cancer Res. Treat., 83: 57-66
Lang, G. A., Iwakuma, T., Suh, Y. A., Liu, G., Rao, V. A., Parant, J. M., Valentin-Vega, Y. A., Terzian, T., Caldwell, L. C., Strong, L. C., et al. (2004). Gain of function of a p53 hot spot mutation in a mouse model of Li-Fraumeni syndrome. Cell 119, 861-872.
Laptenko, O., and Prives, C. (2006). Transcriptional regulation by p53: one protein, many possibilities. Cell Death Differ 13, 951-961.
Larsson, O. (1996). HMG-CoA reductase inhibitors: role in normal and malignant cells. Crit. Rev Oncol Hematol 22, 197-212.
Lerner, E. C., Qian, Y., Blaskovich, M. A., Fossum, R. D., Vogt, A., Sun, J., Cox, A. D., Der, C. J., Hamilton, A. D., and Sebti, S. M. (1995). Ras CAAX peptidomimetic FTI-277 selectively blocks oncogenic Ras signaling by inducing cytoplasmic accumulation of inactive Ras-Raf complexes. J Biol Chem 270, 26802-26806.
Lin, J., Chen, J., Elenbaas, B., and Levine, A. J. (1994). Several hydrophobic amino acids in the p53 amino-terminal domain are required for transcriptional activation, binding to mdm-2 and the adenovirus 5 E1B 55-kD protein. Genes Dev 8, 1235-1246.
Lin, J., Teresky, A. K., and Levine, A. J. (1995). Two critical hydrophobic amino acids in the N-terminal domain of the p53 protein are required for the gain of function phenotypes of human p53 mutants. Oncogene 10, 2387-2390.
Matas, D., Sigal, A., Stambolsky, P., Milyaysky, M., Weisz, L., Schwartz, D., Goldfinger, N., and Rotter, V. (2001). Integrity of the N-terminal transcription domain of p53 is required for mutant p53 interference with drug-induced apoptosis. EMBO J. 20, 4163-4172.
Meyers et al., (1985) Science, Vol. 230, p. 1242.
Mirza, A., Wu, Q., Wang, L., McClanahan, T., Bishop, W. R., Gheyas, F., Ding, W., Hutchins, B., Hockenberry, T., Kirschmeier, P., et al. (2003). Global transcriptional program of p53 target genes during the process of apoptosis and cell cycle progression. Oncogene 22, 3645-3654.
Mitsudomi et al., (1993) Clin Cancer Res 2000 October; 6(10):4055-63; Koshland, Science 262:1953
Mo, H., and Elson, C. E. (2004). Studies of the isoprenoid-mediated inhibition of mevalonate synthesis applied to cancer chemotherapy and chemoprevention. Exp Biol Med (Maywood) 229, 567-585.
Mori, S., Chang, J. T., Andrechek, E. R., Potti, A., and Nevins, J. R. (2009). Utilization of genomic signatures to identify phenotype-specific drugs. PLoS One 4, e6772.
Mullis et al., (1994) The Polymerase Chain Reaction, Birkhauser, Boston.
Muller, P. A., Caswell, P. T., Doyle, B., Iwanicki, M. P., Tan, E. H., Karim, S., Lukashchuk, N., Gillespie, D. A., Ludwig, R. L., Gosselin, P., et al. (2009). Mutant p53 drives invasion by promoting integrin recycling. Cell 139, 1327-1341.
Orita et al., Proc. Natl. Acad. Sci. USA Vol. 86, pp. 2766-2770, 1989.
Orita et al., Proc. Natl. Acad. Sci. Genomics, Vol. 5, pp. 874-879, 1989.
Olive, K. P., Tuveson, D. A., Ruhe, Z. C., Yin, B., Willis, N. A., Bronson, R. T., Crowley, D., and Jacks, T. (2004). Mutant p53 gain of function in two mouse models of Li-Fraumeni syndrome. Cell 119, 847-860.
Olivier, M., Langerod, A., Carrieri, P., Bergh, J., Klaar, S., Eyfjord, J., Theillet, C., Rodriguez, C., Lidereau, R., Bieche, I., et al. (2006). The clinical value of somatic TP53 gene mutations in 1,794 patients with breast cancer. Clin Cancer Res 12, 1157-1167.
Peart, M. J., and Prives, C. (2006). Mutant p53 gain of function: the NF-Y connection. Cancer Cell 10, 173-174.
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. (2000). Molecular portraits of human breast tumours. Nature 406, 747-752.
Petersen, O. W., Ronnov-Jessen, L., Howlett, A. R., and Bissell, M. J. (1992). Interaction with basement membrane serves to rapidly distinguish growth and differentiation pattern of normal and malignant human breast epithelial cells. Proc Natl Acad Sci USA 89, 9064-9068.
Petitjean, A., Achatz, M. I., Borresen-Dale, A. L., Hainaut, P., and Olivier, M. (2007). TP53 mutations in human cancers: functional selection and impact on cancer prognosis and outcomes. Oncogene 26, 2157-2165.
Reed, B. D., Charos, A. E., Szekely, A. M., Weissman, S. M., and Snyder, M. (2008). Genome-wide occupancy of SREBP1 and its partners NFY and SP1 reveals novel functional roles and combinatorial regulation of distinct classes of genes. PLoS Genet. 4, e1000133.
Ruano and Kidd, Nucleic Acids Research, Vol. 17, p. 8392, 1989.
Sato, R. (2010). Sterol metabolism and SREBP activation. Arch Biochem Biophys 501, 177-181.
Savage et al., (2007) Pediatr Blood Cancer; 49:28-33
Shibata, M. A., Ito, Y., Morimoto, J., and Otsuki, Y. (2004). Lovastatin inhibits tumor growth and lung metastasis in mouse mammary carcinoma model: a p53-independent mitochondrial-mediated apoptotic mechanism. Carcinogenesis 25, 1887-1898.
Sorlie, T., Perou, C. M., Tibshirani, R., Aas, T., Geisler, S., Johnsen, H., Hastie, T., Eisen, M. B., van de Rijn, M., Jeffrey, S. S., et al. (2001). Gene expression patterns of breast carcinomas distinguish tumor subclasses with clinical implications. Proc Natl Acad Sci USA 98, 10869-10874.
Stambolsky, P., Tabach, Y., Fontemaggi, G., Weisz, L., Maor-Aloni, R., Siegfried, Z., Shiff, I., Kogan, I., Shay, M., Kalo, E., et al. (2010). Modulation of the vitamin D3 response by cancer-associated mutant p53. Cancer Cell 17, 273-285.
Theophilus et al., “PCR Mutation Detection Protocols,” Humana Press (2002)
Ueda et al., (2006) Gynecol Oncol; 100:173-8
Ugawa, T., Kakuta, H., Moritani, H., Matsuda, K., Ishihara, T., Yamaguchi, M., Naganuma, S., Iizumi, Y., and Shikama, H. (2000). YM-53601, a novel squalene synthase inhibitor, reduces plasma cholesterol and triglyceride levels in several animal species. Br J Pharmacol 131, 63-70.
Varley, Hum Mutat 2003; 21:313-20; Royds et al. Cell Death Differ 2006; 13:1017-26
Vasudevan, A., Qian, Y., Vogt, A., Blaskovich, M. A., Ohkanda, J., Sebti, S. M., and Hamilton, A. D. (1999). Potent, highly selective, and non-thiol inhibitors of protein geranylgeranyltransferase-I. J Med Chem 42, 1333-1340.
Venot, C., Maratrat, M., Siena, V., Conseiller, E., and Debussche, L. (1999). Definition of a p53 transactivation function-deficient mutant and characterization of two independent p53 transactivation subdomains. Oncogene 18, 2405-2410.
Vogelstein, B., Lane, D., and Levine, A. J. (2000). Surfing the p53 network. Nature 408, 307-310.
Wang-Gohrke et al., (1999) Br J. Cancer. 81:179-83
Winter et al., Proc. Natl. Acad. Sci. USA, Vol. 82, p. 7575, 1985
Wong, W. W., Dimitroulakos, J., Minden, M. D., and Penn, L. Z. (2002). HMG-CoA reductase inhibitors and the malignant cell: the statin family of drugs as triggers of tumor-specific apoptosis. Leukemia 16, 508-519.
Wu et al., (1989) Genomics, Vol. 4, pp. 560-569.
Wu et al., (2006) Cancer Res; 66:8287-92.
Yahagi, N., Shimano, H., Matsuzaka, T., Najima, Y., Sekiya, M., Nakagawa, Y., Ide, T., Tomita, S., Okazaki, H., Tamura, Y., et al. (2003). p53 Activation in adipocytes of obese mice. J Biol Chem 278, 25395-25400.
Yan, W., and Chen, X. (2010). Characterization of functional domains necessary for mutant p53 gain of function. J Biol Chem 285, 14229-14238.
Zhang, Y., Yan, W., and Chen, X. (2011). Mutant p53 disrupts MCF-10A cell polarity in 3-dimensional culture via epithelial-to-mesenchymal transitions. J Biol. Chem.

1. A method comprising:
(a) identifying a subject having cancer, precancerous cells, or a benign tumor that has a mutated p53 gene or that expresses a mutant p53 protein or an mRNA encoding a mutant p53 protein; and
(b) administering to the subject a therapeutically effective amount of an SREBP cleavage activating protein inhibitor, in an amount that reduces or eliminates the cancer, the precancerous cells, or the benign tumor.

2. The method of claim 1, wherein step (a) further comprises:
(i) obtaining a biological sample of the cancer, the precancerous cells or the benign tumor from the subject,
(ii) determining if the cancer cells, the precancerous cells or the cells of the benign tumor in the biological sample have the mutant p53 gene, or express a mutant p53 protein or an mRNA encoding a mutant p53 protein, and
(iii) if the mutant p53 gene, or expression of a mutant p53 protein or an mRNA encoding a mutant p53 protein is detected, then identifying the subject as having cancer, precancerous cells, or a benign tumor that has a mutated p53 gene or that expresses a mutant p53 protein or an mRNA encoding a mutant p53 protein.

3. The method of claim 2, wherein the biological sample comprises a tumor biopsy, urine, blood, cerebrospinal fluid, sputum, serum, stool, or bone marrow.

4. The method of claim 2, wherein the cancer is selected from the group consisting of lung cancer, digestive and gastrointestinal cancers, gastrointestinal stromal tumors, gastrointestinal carcinoid tumors, colon cancer, rectal cancer, anal cancer, bile duct cancer, small intestine cancer, stomach (gastric) cancer, esophageal cancer, gall bladder cancer, liver cancer, pancreatic cancer, appendix cancer, breast cancer, ovarian cancer, renal cancer, cancer of the central nervous system, skin cancer, lymphomas, choriocarcinomas, head and neck cancer, osteogenic sarcomas, and blood cancers.

5. The method of claim 2, wherein the cancer is breast cancer.

6. The method of claim 2, wherein the SREBP cleavage activating protein inhibitor is fatostatin or an analogue thereof.

7. The method of claim 6, wherein the amount of fatostatin or an analogue thereof ranges from 0.1 mg/kg to about 150 mg/kg.

8. The method of claim 7, wherein the amount of fatostatin or an analogue thereof is about 10 mg/kg to about 50 mg/kg.

9. The method of claim 6, wherein the fatostatin or an analogue thereof is administered orally, by injection, parenterally, by inhalation spray, topically, rectally, nasally, buccally, vaginally, or via an implanted reservoir.

10. The method of claim 6, wherein fatostatin or an analogue thereof is administered locally to the site of the cancer, the precancerous cell, or the benign tumor.

11. The method of claim 6, wherein fatostatin or an analogue thereof is administered alone, or in combination with a statin.

12. The method of claim 11, wherein the statin is selected from the group consisting of lovastatin, simvastatin, pravastatin, fluvastatin, atorvastatin, and cerivastatin.

13. A method for determining if cancer or precancerous lesions or benign tumors in a subject will be responsive to treatment with a SREBP cleavage activating protein inhibitor, comprising:
(a) obtaining a biological sample of cells from the cancer, the precancerous lesions or the benign tumors from the subject;
(b) assaying the cells in the sample for the presence of a mutated p53 gene or expression of a mutant form of p53 protein or a biologically active fragment thereof or an mRNA encoding the mutant form of p53 protein, and;
(c) if the mutated p53 gene or the mutant form of the p53 protein or the mRNA encoding the mutant form of p53 protein is detected in the cells, then determining that the cancer, the precancerous lesions, the benign tumors will respond to treatment with the inhibitor.

14. A method for preventing recurrence of cancer, precancerous lesions or a benign tumor having a mutated p53 gene or expressing a mutant form of p53 protein or a biologically active fragment thereof or an mRNA encoding the mutant form of p53 protein in a subject, comprising administering to the subject a prophylactically effective amount of an SREBP cleavage activating protein inhibitor.

15. A method of preventing cancer in a subject at high risk of developing a form of cancer that expresses a mutant p53 protein or a p53 gene mutation or an mRNA encoding a mutant form of p53 protein, comprising administering to the subject an SREBP cleavage activating protein inhibitor in a prophylactically effective amount.

16. The method of claim 14 wherein the SREBP cleavage activating protein inhibitor is fatostatin or an analogue thereof.

17. The method of claim 15 wherein the SREBP cleavage activating protein inhibitor is fatostatin or an analogue thereof.

18. A pharmaceutical composition comprising therapeutically effective amounts of fatostatin or an analogue thereof in a range of from about 0.1 mg to about 150 mg.

19. The pharmaceutical composition of claim 18, wherein the amount of fatostatin is 30 mg/kg.

20. A pharmaceutical composition comprising therapeutically effective amounts of fatostatin or an analogue thereof in combination with one or more statins.

21. The pharmaceutical composition of claim 20, wherein the statin is selected from the group consisting of lovastatin, simvastatin, pravastatin, fluvastatin, atorvastatin, and cerivastatin.

22. The pharmaceutical composition of claim 20, wherein said statin is in an amount between less than about 80 mg/day.

23. A kit containing the pharmaceutical composition of claim 18.

24. The method of claim 1, wherein the subject is a human.
